(19) United States
(12) Patent Application Publication
(10) Pub. No.: US 2026/0049059 A1
(43) Pub. Date: Feb. 19, 2026

(54) A PROCESS FOR PREPARATION OF CABOZANTINIB OR TIVOZANIB

(71) Applicant: Synthon B.V., Nijmegen (NL)

(72) Inventors: Ondrej HYLSE, Brnenska (CZ); Jiri PARTL, Brnenska (CZ)

(21) Appl. No.: 19/132,542

(22) PCT Filed: Nov. 23, 2023
(86) PCT No.: PCT/EP2023/082902

§ 371 Date: May 23, 2025

(30) Foreign Application Priority Data
Nov. 25, 2022 (EP) 22209650.5

(51) Int. Cl. C07D 215/233; C07D 413/12

(52) U.S. Cl. CPC C07D 215/233; C07D 413/12

(57) ABSTRACT

The presented invention relates to a process for preparation of Cabozantinib, compound of formula (1), or Tivozanib, compound of formula (10) or a salr or a solvate thereof: Formula (1); Formula (10).

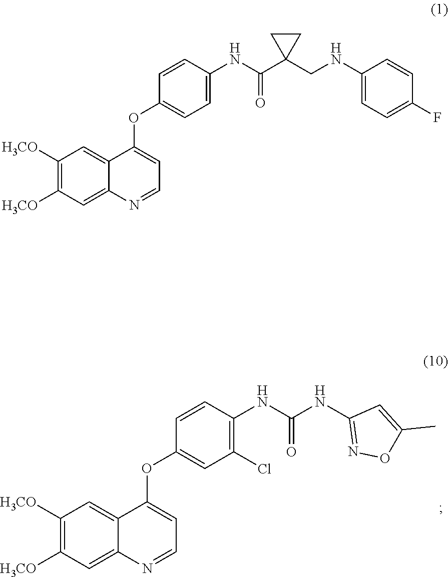 [SMILES: COc1cc2nccc(Oc3ccc(NC(=O)C4(CNc5ccc(F)cc5)CC4)cc3)c2cc1OC ; COc1cc2nccc(Oc3ccc(NC(=O)Nc4cc(C)on4)c(Cl)c3)c2cc1OC]

### BACKGROUND OF THE PRESENT INVENTION

The presented invention relates to a process for preparation of Cabozantinib, compound of formula (1), or Tivozanib, compound of formula (10) or a salt or a solvate:

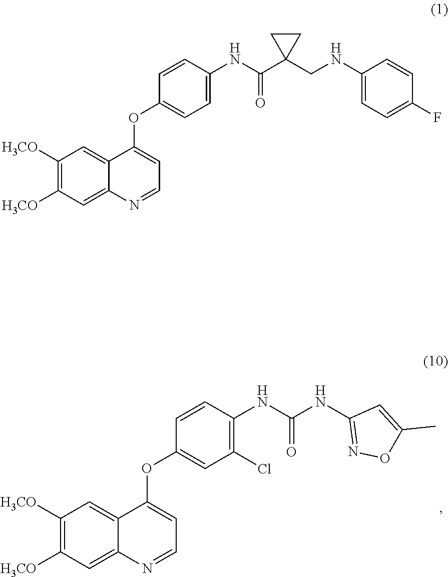 [SMILES: COc1cc2nccc(Oc3ccc(NC(=O)C4(CNc5ccc(F)cc5)CC4)cc3)c2cc1OC ; COc1cc2nccc(Oc3ccc(NC(=O)Nc4cc(C)on4)c(Cl)c3)c2cc1OC]

thereof comprising:
a. Reacting compound of formula (2) with compound of formula (3) in a solvent selected from N,N-dimethylformamide or N,N-dimethylacetamide or N-methyl pyrrolidone to obtain compound of formula (4):

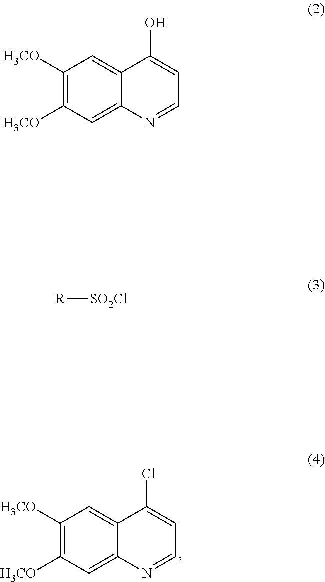

R is selected from C₁ to C₇ linear or branched optionally substituted alkyl or aryl;
b. Transforming compound of formula (4) into Cabozantinib or Tivozanib.

Cabozantinib, N-[4-(6,7-Dimethoxyquinolin-4-yloxy)phenyl]-N′-(4-fluorophenyl) cyclopropane-1,1-dicarboxamide, is an orally bioavailable antineoplastic agent approved for the treatment of unresectable, locally advanced or metastatic medullary thyroid cancer. Cabozantinib was first disclosed in WO2005030140 application. The processes for preparation of Cabozantinib are described for example in applications WO2005030140 or WO2010036831.

Tivozanib, N-[2-Chloro-4-(6,7-dimethoxyquinolin-4-yloxy)phenyl]-N′-(5-methylisoxazol-3-yl)urea, is a small-molecule inhibitor of vascular endothelial growth factor receptors (VEGFR) 1, 2 and 3 approved as a first-line treatment for adult patients with advanced renal cell carcinoma (RCC), as well as for the treatment of adults with advanced RCC who are VEGFR and mTOR pathway inhibitor-naïve, following disease progression after one prior treatment with cytokine therapy. Tivozanib was first disclosed in WO2002088110 application. The processes for preparation of Tivozanib are described for example in applications WO2002088110 or WO2004035572.

In the processes for preparation of both Cabozantinib and Tivozanib compound of formula (4) is used as an intermediate. The processes for preparation thereof described in prior art mostly use POCl₃ in a process for preparation of compound of formula (4). POCl₃ is highly toxic compound, it releases 3 equivalents of HCl and 1 equivalent of H₃PO₄ upon workup of the reaction mixture, additionally to that the workup is exotermic and requires significant cooling of the reaction mixture and subsequent neutralization of released acids with high amount of solutions of bases. That all causes the use of POCl₃ highly impractical and there is a need to replace POCl₃ with other agent that is not toxic with better processability of reaction mixture.

### BRIEF DESCRIPTION OF THE PRESENT INVENTION

The presented invention relates to a process for preparation of Cabozantinib, compound of formula (1), or Tivozanib, compound of formula (10),

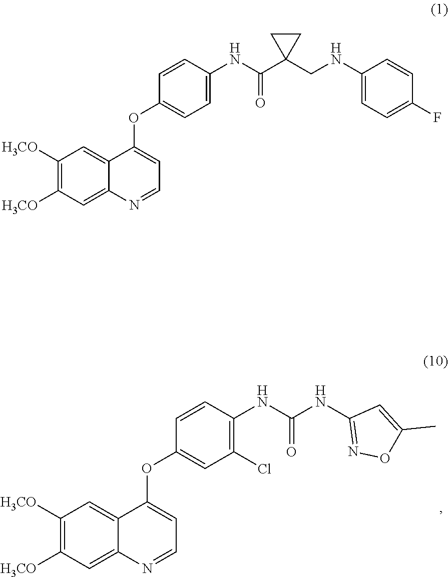 [SMILES: COc1cc2nccc(Oc3ccc(NC(=O)C4(CNc5ccc(F)cc5)CC4)cc3)c2cc1OC ; COc1cc2nccc(Oc3ccc(NC(=O)Nc4cc(C)on4)c(Cl)c3)c2cc1OC]

the process comprising:
a. Reacting compound of formula (2) with compound of formula (3) in a solvent selected from N,N-dimethylformamide or N,N-dimethylacetamide or N-methyl pyrrolidone to obtain compound of formula (4):

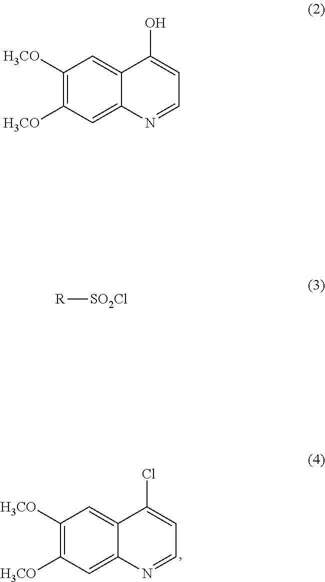

R is selected from C₁ to C₇ linear or branched optionally substituted alkyl or aryl;
b. Transforming compound of formula (4) into Cabozantinib or Tivozanib

### BRIEF DESCRIPTION OF THE DRAWINGS

FIG. 1: XRPD pattern of compound of formula (4) prepared according to Example 1 or Example 1 or Example 2

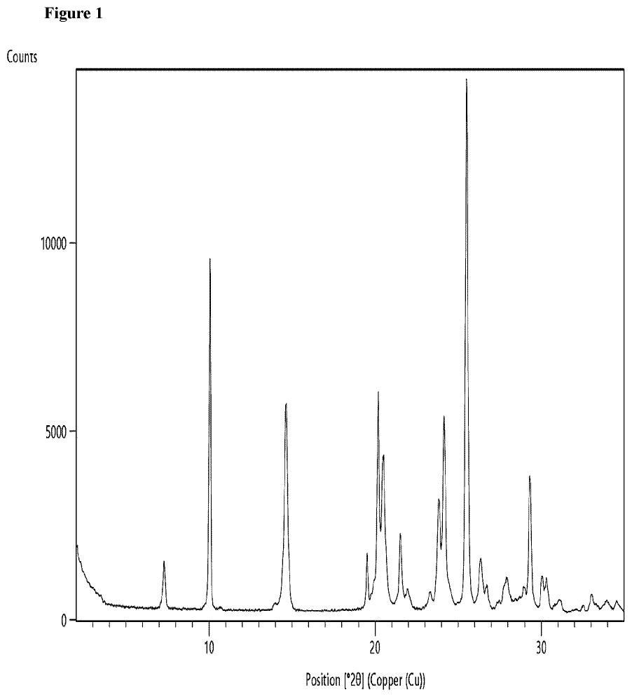

FIG. 2: XRPD pattern of compound of formula (6) prepared according to Example 3

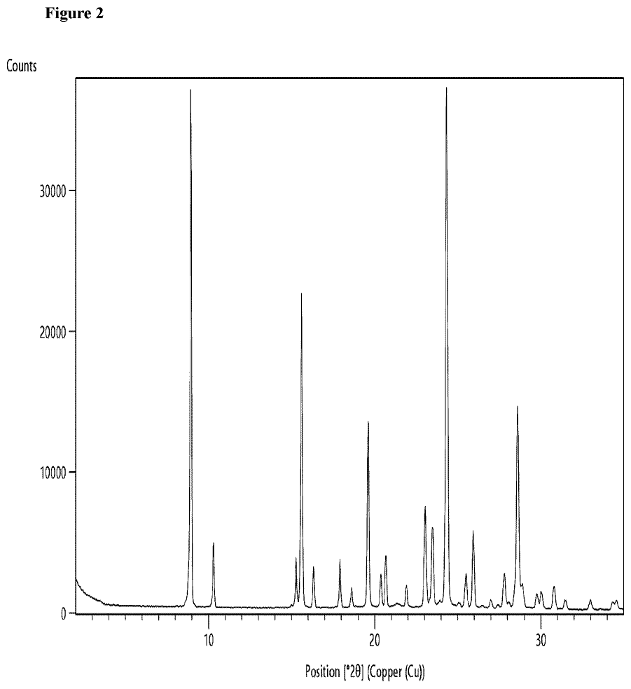

FIG. 3: XRPD pattern of compound of formula (7) prepared according to Examples 4 to 7

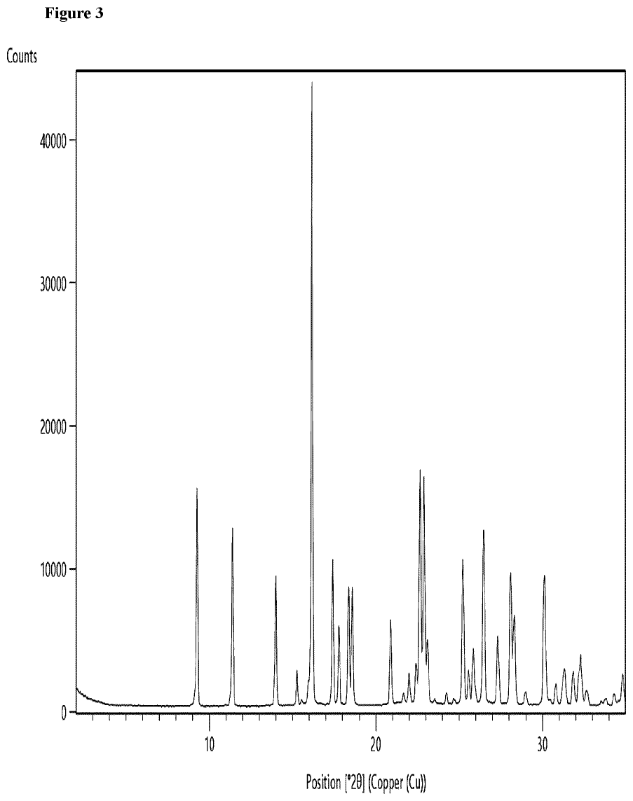

FIG. 4: XRPD pattern of compound of formula (13) prepared according to Example 12

### DETAILED DESCRIPTION OF THE INVENTION

The presented invention relates to a process for preparation of Cabozantinib, compound of formula (1), or Tivozanib, compound of formula (10):

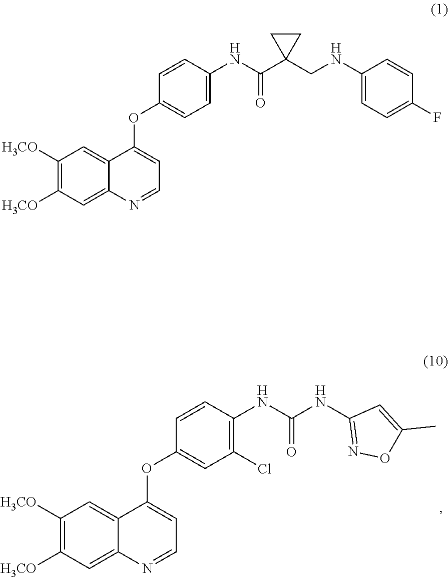 [SMILES: COc1cc2nccc(Oc3ccc(NC(=O)C4(CNc5ccc(F)cc5)CC4)cc3)c2cc1OC ; COc1cc2nccc(Oc3ccc(NC(=O)Nc4cc(C)on4)c(Cl)c3)c2cc1OC]

the process comprising:
a. Reacting compound of formula (2) with compound of formula (3) in a solvent selected from N,N-dimethylformamide or N,N-dimethylacetamide or N-methyl pyrrolidone to obtain compound of formula (4):

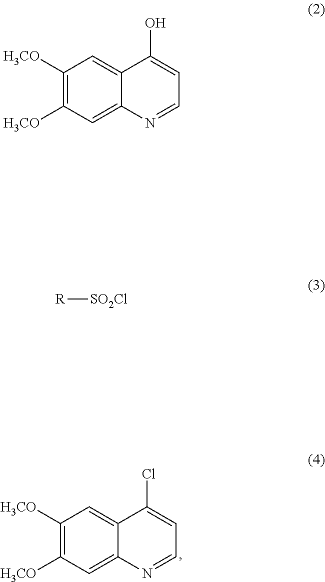

R is selected from C₁ to C₇ linear or branched optionally subsituted alkyl or aryl;
b. Transforming compound of formula (4) into Cabozantinib or Tivozanib.

The solvent in step a. can be selected from N,N-dimethylformamide or N,N-dimethylacetamide or N-methyl pyrrolidone, preferably it is N,N-dimethylformamide.

R in compound of formula (3) is selected from C₁ to C₇ optionally subsituted alkyl or aryl for example from CH₃ or CH₃CH₂,

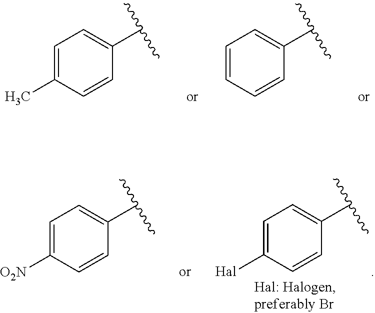

The compound of formula (3) can be selected for example from benzenesulfonyl chloride or 4-methylbenzene sulfonyl chloride or methanesulfonyl chloride or ethanesulfonyl chloride, preferably it is selected from benzenesulfonyl chloride or 4-methylbenzene sulfonyl chloride.

The concentration of compound (2) in the solvent can be between 0.1 g/ml and 0.5 g/ml, preferably it is between 0.1 g/ml and 0.3 g/ml. The concentration of compound of fromula (3) in the solvent can be between 0.1 g/ml and 0.5 g/ml, preferably it is between 0.1 g/ml and 0.3 g/ml. The molar ratio beween compound of formula (2) and compound of formula (3) can be between 1:1 and 1:2, preferably it is beween 1:1 and 1:1.5. Compound of formula (2) is mixed with the solvent. The presence of water reaction between compound (2) and compound (3) can influence the reaction yield and purity of prepared compound of formula (4). In the case that water is present for exaple in used solvent or compound of formula (2) the amount of water in the reaction mixture can be decreased for example by distilling off a part of the solvent, for example 1/10 (vol) or ⅕ (vol) or ⅓ (vol) of the solvent and replacing with the same amount of the solvent selected from N,N-dimethyl-formamide or N,N-dimethylacetamide or N-methyl pyrrolidone. The amount of water can be also decreased using a drying agent for example magnesium sulfate.

To the mixture a base is added. The base can be selected form an organic base for example triethylamine or diethyl amine or a tertiary amine. The molar ratio between compound of formula (2) and the base can be between 1:1.1 and 1:2, preferably it is between 1:1.1 and 1:1.5. To the mixture, compound of formula (3) is added. The reaction mixture is heated to a temperature between 70° C. and 90° C. and stirred at this temperature for between 1.5 and 10 hours, preferably for between 2 and 4 hours. The reaction progress can be monitored by any suitable analytical method for example by HPLC or GC. After the reaction is completed, the mixture is cooled to a temperature between 10° C. and 55° C., preferably between 20° C. and 50° C. To the mixture a water solution of a base is added. A base can be selected from for example a hydroxide, such as sodium hydroxide or potassium hydroxide or a carbonate such as sodium carbonate of potassium carbonate. The concentration of the base in water can be between 0.1 g/ml and 0.5 g/ml, preferably it is between 0.1 and 0.3 g/ml. The molar ratio between the base and the compound of formula (1) can be between 1:0.5 and 1:5, preferably it is between 1:0.6 and 1:3. The mixture is stirred at a temperature between 10° C. and 55° C., preferably between 20° C. and 50° C. for between 0.5 and 5 hours to obtain a suspension. Obtained suspension was filtered, obtained solid can be optionally washed with water or an organic solvent such as heptane and dried. Obtained solid form of compound of formula (4) can be characterized by XRPD pattern having 20 values 10.1°, 20.2° and 25.5° degrees 2 theta (±0.2 degrees 2 theta) when measured with CuKα1 radiation (Δ=1.54060 Å).

Obtained solid form of compound of formula (4) can be also characterized by XRPD pattern described in following Table:

| Angle (2-Theta °) | Intensity (%) |
|---|---|
| 7.3 | 10.8 |
| 10.1 | 67.9 |
| 14.0 | 3.1 |
| 14.6 | 40.5 |
| 19.5 | 12.2 |
| 20.2 | 42.3 |
| 20.5 | 30.4 |
| 21.5 | 15.9 |
| 22.0 | 5.7 |
| 23.3 | 5.1 |
| 23.8 | 22.5 |
| 24.2 | 38.2 |
| 25.5 | 100.0 |
| 26.4 | 11.4 |
| 26.7 | 6.5 |
| 27.4 | 3.6 |
| 27.9 | 7.9 |
| 29.0 | 6.2 |
| 29.3 | 26.8 |
| 30.0 | 8.2 |
| 30.3 | 7.8 |
| 31.1 | 3.8 |
| 32.5 | 2.7 |
| 33.0 | 4.6 |
| 33.9 | 3.6 |
| 34.5 | 3.6 |

Transforming the compound of formula (4) into Cabozantinib, compound of formula (1), can be done for example by a process described in the prior art or by a process comprising:
a. Reacting compound of formula (4) with compound of formula (5) to obtain compound of formula (6):

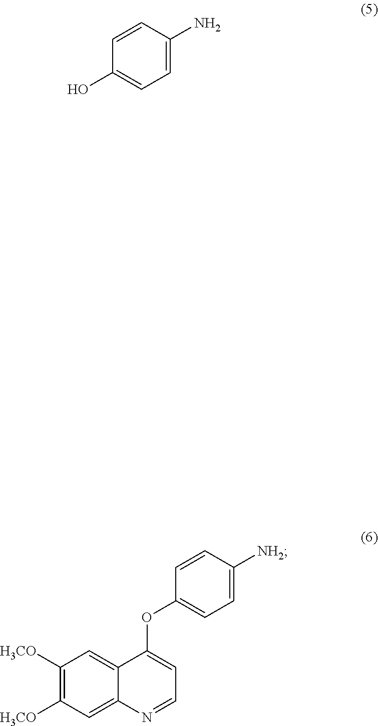 [SMILES: Nc1ccc(O)cc1 ; COc1cc2nccc(Oc3ccc(N)cc3)c2cc1OC]

b. Reacting compound of formula (6) with compound of formula (7) to obtain compound of formula (1):

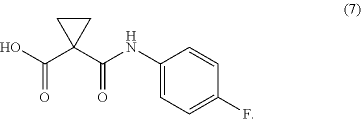 [SMILES: O=C(O)C1(C(=O)Nc2ccc(F)cc2)CC1]

The reaction between compound od formula (4) and compound of formula (5) can be done in a solvent selected for example from N,N-dimethylacetamide or N,N-dimethyl-formamide. The concentration of compound of formula (4) can be between 0.05 g/ml and 0.3 g/ml. The concentration of compound of formula (5) in the solvent can be between 0.05 g/ml 15 and 0.3 g/ml. The molar ratio between compound of formula (4) and compound of formula (5) can be between 1:1.1 and 1:3, preferably it is between 1:1.3 and 1:2. The reaction is done in a presence of a base. The base can be selected from for example a hydroxide, such as sodium hydroxide or potassium hydroxide. The molar ratio between compound of formula (4) and the base can be between 1:1.2 and 1:3, preferably between 1:1.3 and 1:2.

Compound of formula (4), compound of formula(5) and the base are mixed with the solvent. The mixture was heated to a temperature between 90° C. and 130° C. and stirred at this temperature for between 1.5 and 5 hours. The reaction progress can be monitored by any suitable analytical method for example by HPLC or GC. After the reaction is completed, the mixture was cooled to a temperature between 35° C. and 45° C. and to the mixture methanol and water solution of a base was added. The base can be selected from a hydroxide for example sodium hydroxide or potassium hydroxide. The concentration of the base in the water solution of the base can be between 0.007 g/ml and 0.012 g/ml. The molar ratio between the base and the compound of formula (4) can be between 2:1 and 3:1, preferably it is between 2.4:1 and 2.8:1. Addition of water solution of a base improves purity and processability of obtained compound of formula (6). The volume ratio between methanol and the water base solution can be between 1:2 and 1:3, preferably it is between 1:2.2 and 1:2.6. The volume ratio between methanol and the solvent used in reaction between compound (4) and compound (5) can be between 1:2 and 1:3, preferably it is between 1:2.3 and 1:2.7. After addition of methanol and water base solution the mixture can be stirred for example for between 5 and 60 minutes at a temperature between 30° C. and 50° C. The mixture can be then cooled to a temperature between 10° C. and 25° C. and stirred at this temperature for between 30 and 240 minutes to obtain a suspension. The suspension is filtered off and obtained solid can be optionally washed with a solvent such as N,N-dimethylacetamide or tetrahydrofurane or mixture thereof with water. Obtained solid form of compound of formula (6) can be characterized by XRPD pattern having 20 values 8.9°, 15.6° and 24.3° degrees 2 theta (±0.2 degrees 2 theta) when measured with CuKα1 radiation (Δ=1.54060 Å).

Obtained solid form of compound of formula (6) can be also characterized by XRPD pattern described in following Table:

| Angle (2-Theta °) | Intensity (%) |
|---|---|
| 8.9 | 98.6 |
| 10.3 | 13.1 |
| 15.0 | 1.5 |
| 15.3 | 10.4 |
| 15.6 | 62.1 |
| 16.3 | 9.4 |
| 17.9 | 10.2 |
| 18.6 | 4.8 |
| 19.6 | 37.6 |
| 20.4 | 7.3 |
| 20.7 | 11.0 |
| 21.4 | 1.8 |
| 21.9 | 5.2 |
| 23.1 | 20.0 |
| 23.5 | 16.4 |
| 23.9 | 2.5 |
| 24.3 | 100.0 |
| 25.1 | 2.0 |
| 25.5 | 7.4 |
| 25.9 | 15.5 |
| 26.5 | 1.4 |
| 27.0 | 2.6 |
| 27.4 | 1.5 |
| 27.8 | 7.4 |
| 28.1 | 2.0 |
| 28.6 | 39.3 |
| 28.9 | 5.3 |
| 29.8 | 3.6 |
| 30.0 | 4.0 |
| 30.8 | 4.9 |
| 31.5 | 2.5 |
| 33.0 | 2.5 |
| 34.3 | 2.1 |
| 34.5 | 2.3 |

The compound of formula (6) is then reacted with compound of formula (7) to obtain Cabozantinib, compound o formula (1) by a process disclosed in the prior art, for example in W02012/1095 10 application or by a process comprising reaction of compound (6) and compound (7) in a solvent selected for example from tetrahydrofurane or 2-methyl tetra-hydrofurane in a presence of a coupling agent such as CDMT (2-Chloro-4,6-dimethoxy-1,3,5-triazine) or EDC (1-ethyl-3-(3-dimethylaminopropyl)carbodiimide) or DCC (N,N′-Dicyclohexylcarbodiimide) or T3P (Propanephosphonic acid anhydride). The molar ratio between compound (6) and compound (7) can be between 1:1.05 and 1:2, preferably it is between 1:0.5 and 1:1.5. The concentration of compound of formula (6) in the solvent can be between 0.03 g/ml and 0.3 g/ml, preferably it is between 0.03 g/ml and 0.1 g/ml. The concentration of compound of formula (7) in the solvent can be between 0.03 g/ml and 0.3 g/ml, preferably it is between 0.03 g/ml and 0.1 g/ml. The concentration of the coupling agent in the solvent can be between 0.03 g/ml and 0.3 g/ml, preferably it is between 0.03 g/ml and 0.1 g/ml. The molar ratio between the compound of formula (6) and the coupling agent can be between 1:1.05 and 1:2, preferably it is between 1:0.5 and 1:1.5. Compound of formula (6) is mixed with compound of formula (7), the coupling agent and the solvent. The mixture is then stirred for example at a temperature between 20° C. and 60° C. for between 2 and 10 hours.

The reaction progress can be monitored by any suitable analytical method for example by HPLC or TLC. After the reaction is completed, the mixture can be washed for example with water or water solution of a base at a temperature between 20° C. and 80° C. The phases are separated and the organic phase can be optionally treated with for example active carbon. The organic phase can be evaporated to obtain Cabozantinib, compound of formula (1).

Cabozantinib, compound of formula (1), can be also prepared by a process comprising:
a. Reacting compound of formula (7) with a acyl-chloride forming agent such as thionyl chloride or oxalyl dichloride in a suitable solvent, such as tetrahydro-furane or 2-methyl tetrahydrofurane to obtain a reaction mixture;
b. Adding the mixture into a mixture of compound of formula (6) and a base in a suitable solvent such as tetrahydrofurane or 2-methyl tetrahydrofurane and water.

The concentration of formula (7) in the solvent can be between 0.05 g/ml and 0.15 g/ml. The molar ratio between compound of formula (7) and the acyl-chloride forming agent can be between 1:1.2 and 1:1.6, preferably it is between 1:1.3 and 1:1.5. Compound of formula (7) is mixed with acyl chloride forming agent and the solvent. The mixture is stirred at a temperature between 55° C. and 65° C. for between 1.5 and 5 hours to obtain a mixture.

Compound of formula (6) is mixed with the solvent and the base to obtain a mixture. The solvent is preferably a solvent immiscible with water. The base is preferably added in a form of water solution. The base can be selected form a hydroxide such as sodium hydroxide or potassium hydroxide or a carbonate such as sodium carbonate or potassium carbonate. The molar ratio between the base and the compound of formula (6) can be between 2:1 and 7:1, preferably it is between 3:1 and 4:1.

The mixture obtained in step a is added to the mixture in step b. in several portions, preferably dropwise in the course of between 5 and 60 minutes at a temperature between 20° C. and 50° C. After the addition, the phases are separated and the organic phase can be washed for example with saturated water solution of NaCl. Compound of formula (1) can be isolated for example by distilling off the solvent.

Compound of formula (7) can be prepared by a process comprising:
a. Mixing compound of formula (8) with an acyl chloride forming agent in a solvent to obtain a mixture:

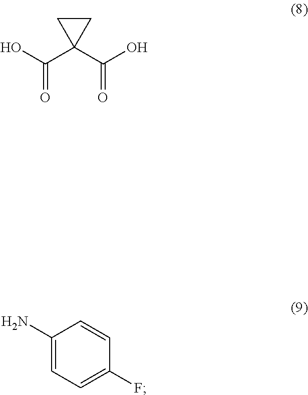 [SMILES: O=C(O)C1(C(=O)O)CC1 ; Nc1ccc(F)cc1]

b. Adding the mixture into a two-phases system of a water immiscible solvent, a water solution of a base and compound of formula (9);
c. Setting pH of a mixture obtained in step b. between 11 and 13;
d. Separating the phases;
e. Setting pH of water phase obtained in step d. between 1 and 2 to provide compound of formula (7).

In the step a. the acyl chloride forming agent can be selected for example from thionyl chloride or oxalyl dichloride. The solvent can be selected for example from an ether such as methyl tert-butyl ether. The molar ratio between compound of formula (8) and formula (9) can be between 1:1.1 and 1:2, preferably it is between 1.1.1 and 1:1.5. The molar ratio between compound of formula (8) and the acyl chloride forming agent can be between 1:1 and 1:2, preferably it is between 1:1.1 and 1:1.5. The acyl chloride forming agent is mixed with the solvent and compound of formula (8) at a temperature between −10° C. and 50° C., preferably at a temperature between 35° C. and 45° C. The concentration of compound of formula (8) in the solvent can be between 0.1 g/ml and 0.8 g/ml, preferably it is between 0.3 g/ml and 0.6 g/ml. The concentration of the acyl chloride forming agent in the solvent can be between 0.2 g/ml and 0.8 g/ml, preferably it is between 0.4 g/ml and 0.6 g/ml. The mixture is stirred at a temperature between −10° C. and 50° C., preferably at a temperature between 35° C. and 45° C., for between 30 and 240 minutes. Obtained mixture was added preferably in several portions (for example 2 or 3 or 4 or 5 or 6 or 7 portions, preferably dropwise) to a cooled mixture, preferably two-phases mixture, of a solvent, preferably solvent non-miscible with water, compound of formula (9) and water solution of a base. The mixture is cooled to a temperature between −10° C. and 10° C. The solvent, preferably non-miscible with water solvent, can be selected for example from an ether such as methyl-tert butyl ether or tetra-hydrofurane. The concentration of compound of formula (9) can be between 0.1 g/ml and 0.8 g/ml, preferably it is between 0.3 g/ml and 0.6 g/ml. The base can be selected from for example a hydroxide such as sodium and potassium hydroxide or a carbonate such as sodium carbonate or potassium carbonate. The concentration of the base in water solution of the base can be between 0.1 g/ml and 0.5 g/ml, preferably it is between 0.1 g/ml and 0.3 g/ml. To obtain mixture a water solution of a base was added to set the pH of the mixture between 11 and 13, preferably to 12. The base can be selected from for example a hydroxide such as sodium or potassium hydroxide or a carbonate such as sodium carbonate or potassium carbonate. The concentration of the base in the water solution of the base can be between 3 mol/1 and 7 mol/l, preferably it is between 4 mol/1 and 6/mol/l. The phases are separated. To obtained water phase an acid is added to set pH of the mixture between 1 and 2 to obtain a suspension. The acid can be selected for example from HCl or trifluoroacetic acid, preferably 35% water HCl solution is used. Obtained suspension is cooled to a temperature between 0° C. and 5° C. and filtered off. Obtained solid can be optionally washed and dried.

The processes described in prior art are exothermic and provide reaction mixture as a thick suspension difficult to stirred and processed. The process according to presented invention is much less exothermic and therefore more suitable for high scale production. Additionally to that obtained reaction mixture is a thin, well stirable and processable suspension.

The compound of formula (7) can be also prepared by a process described in the prior art or by a process comprising reacting compound of formula (8) and compound of formula (9):

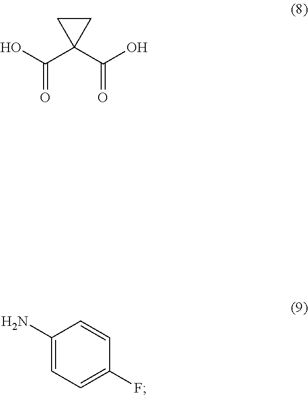 [SMILES: O=C(O)C1(C(=O)O)CC1 ; Nc1ccc(F)cc1]

in a solvent in presence of a acyl chloride forming agent selected for example from thionyl chloride or oxalyl dichloride. The solvent can be selected for example from 2-methyl tetrahydrofurane or methyl tert-butyl ether. The molar ratio between compound (8) and compound (9) can be between 1:1 and 1.5:1, preferably it is between 1:1 and 1.2:1. The concentration of compound (8) in the solvent can be between 0.1 g/ml and 0.8 g/ml. The concentration of compound (9) in the solvent can be between 0.1 g/ml and 0.8 g/ml, preferably it is between 0.3 g/ml and 0.6 g/ml. The molar ratio between compound of formula (8) and the acyl chloride forming agent can be between 1:1 and 1:2, preferably it is between 1:1 and 1:1.3. Compound (8) is mixed with the solvent, the mixture can be optionally cooled, for example to a temperature between −10° C. and 10° C. To the mixture the acyl chloride forming agent is added. The acyl chloride forming agent can be added in portions, for example in 2 or 3 or 5 or 6 or 7 or 8 portions, more preferably dropwise. The mixture is stirred at this temperature for between 1 and 5 hours. To obtained mixture a mixture of compound (9) in the solvent is added at a temperature between −10° C. and 30° C., preferably in portions, for example in 2 or 3 or 5 or 6 or 7 or 8 portions, more preferably dropwise. The mixture is heated to a temperature between −10° C. and 30° C. for between 30 and 240 minutes. The reaction progress can be monitored by any suitable analytical method for example by HPLC or GC. After the reaction is completed, to the mixture a water solution of a base is added at a temperature between 0° C. and 30° C. The base can be selected from for example a hydroxide such as sodium hydroxide or potassium hydroxide or a carbonate such as sodium carbonate or potassium carbonate. The concentration of the base in water can be selected from 0.1 g/ml and 0.5 g/ml. The temperature of the mixture is set to between 20° C. and 30° C. and the mixture can be optionally stirred at this temperature for between 5 and 60 minutes.

The mixture can be processed for example by a process comprising:
i. The phases are separated and the organic phase is washed for example with water or water solution of NaCl, for example 10% NaCl water solution. Phases are separated and to the organic phase a solvent for example toluene or dioxane or 2-butanol is added. The mixture is evaporated, approx. 50% (vol) of solvent is evaporated to obtain a suspension. Obtained suspension was cooled to a temperature between −10° C. and 10° C. and stirred at this temperature for between 1 and 5 hours. The suspension was filtered off and obtained solid can be optionally washed and dried, or by a process comprising:;
ii. The phases are separated and the organic phase is washed with a water solution of a base, for example sodium or potassium hydroxide. The concentration of water solution of a base can be between 0.5 mol/l and 2 mol/l. The phases are separated and to to the water phase an acid is added to a final pH between 1 and 4 to obtain a suspension. The acid can be selected for example from HCl or trifluoroacetic acid. The suspension is filtered off and obtained solid can be optionally washed with for example water solution of an acid such as HCl and dried.

Compound of formula (7) can be purified by a process comprising:
a. Mixing compound of formula (7) in a mixture of isopropyl acetate and water;
b. Separating the phases;
c. Isolating the solid form of compound of formula (7).

The volume ration between water and isopropylacetate can be between 1:2 and 1:2.5, preferably it is between 1:2.2 and 1:2.4. The concentration of compound of formula (7) in the mixture can be between 0.02 g/ml and 0.07 g/ml. Compound of formula (7) is dissolved in the mixture of water and isopropylacetate and the mixture is stirred for between 5 and 60 minutes at a temperature between 20° C. and 25° C. The phases are separated and the organic phase is dried for example using MgSO₄. The organic phase is concentrated to approx. 50% (vol) of the original volume until a suspension was obtained. Obtained suspension is then stirred at a temperature between 20° C. and 25° C. for between 3 and 10 hours. Then the suspension is filtrated off and obtained solid compound of formula (7) can be optionally dried. Obtained solid form of compound of formula (7) can be characterized by XRPD pattern having 20 values 9.3°, 16.2° and 22.7° degrees 2 theta (±0.2 degrees 2 theta) when measured with CuKα1 radiation (λ=1.54060 Å).

Obtained solid form of compound of formula (7) can be also characterized by XRPD pattern described in following Table:

| Angle (2-Theta °) | Intensity (%) |
|---|---|
| 9.3 | 35.7 |
| 11.4 | 30.6 |
| 14.0 | 22.8 |
| 15.3 | 6.5 |
| 15.5 | 1.9 |
| 15.9 | 5.1 |
| 16.2 | 100.0 |
| 17.4 | 24.1 |
| 17.8 | 13.4 |
| 18.4 | 20.0 |
| 18.6 | 20.6 |
| 20.9 | 14.6 |
| 21.7 | 3.0 |
| 22.0 | 6.2 |
| 22.4 | 7.6 |
| 22.7 | 38.6 |
| 22.9 | 36.9 |
| 23.1 | 11.3 |
| 23.5 | 2.0 |
| 24.2 | 2.9 |
| 24.7 | 2.1 |
| 25.2 | 24.3 |
| 25.6 | 6.5 |
| 25.9 | 10.0 |
| 26.5 | 29.3 |
| 27.3 | 11.9 |
| 28.1 | 21.9 |
| 28.3 | 15.0 |
| 29.0 | 3.1 |
| 30.1 | 21.5 |
| 30.8 | 4.3 |
| 31.3 | 6.7 |
| 31.8 | 6.4 |
| 32.2 | 6.9 |
| 32.3 | 9.6 |
| 32.7 | 3.4 |
| 33.5 | 1.6 |
| 33.8 | 2.1 |
| 34.3 | 2.8 |
| 34.8 | 5.9 |

Compound of formula (4) can be transformed into Tivozanib, compound of formula (10) by a process described in the prior art, for example in EP1559715 application, or by a process comprising:
a. Reacting compound of formula (4) with compound of formula (11) or a salt thereof to obtain compound of formula (12) or a salt thereof,

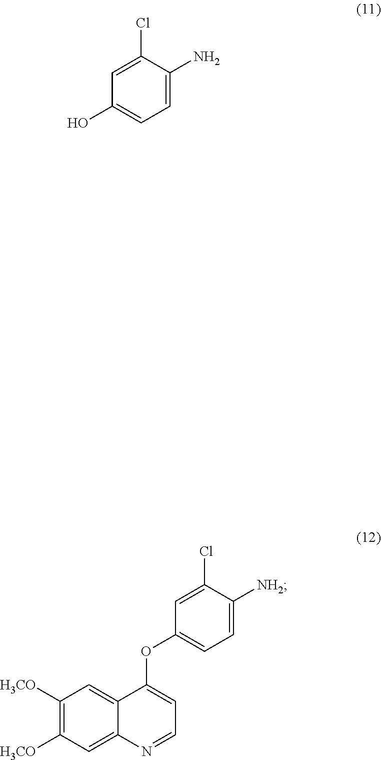 [SMILES: Nc1ccc(O)cc1Cl ; COc1cc2nccc(Oc3ccc(N)c(Cl)c3)c2cc1OC]

b. Compound of formula (12) is reacted with compound of formula (13) to obtain Tivozanib, compound of formula (10), a salt or a solvate thereof,

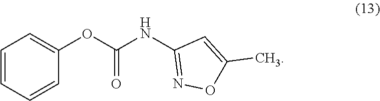 [SMILES: Cc1cc(NC(=O)Oc2ccccc2)no1]

Compound of formula (4) can be reacted with compound of formula (11) in a solvent selected for example from N,N-Dimethylacetamide or N,N-Dimethylformamide or N-Methyl pyrollidone. The concentration of compound of formula (4) in the solvent can be between 0.05 g/ml and 0.25 g/ml. The concentration of compound of formula (11) in the solvent can be between 0.1 g/ml and 0.3 g/ml. The molar ration between compound of formula (4) and compound of formula (11) can be between 0.45:1 and 1:1. Compound of formula (11) can we used in a form of a salt, for example HCl salt. The reaction is perform in a presence of a base, for example an amine such as dimethylamine or trimethylamine. The concentration of the base in the solvent can be between 0.05 g/ml and 0.2 g/ml. Molar ratio between the compounf of formula (4) and the base can be between 0.45:1 and 1:1. Compound of formula (11) or a salt thereof is mixed with the solvent to obtain a solution. The solution is cooled to a temperature between −5° C. and 10° C., preferably to a temperature between −5° C. and 5° C. To the mixture the base is added. The base is preferably added in portions, for example in 3 or 4 or 5 or 6 or 7 or 8 portions. In the case the base is a liquid, it can be added dropwise in the course between 5 and 20 minutes. The mixture is then cooled to a temperature between −5° C. and 10° C., preferably to a temperature between −5° C. and 5° C. and stirred at this temperature for between 15 and 40 minutes. Obtained suspension can be optionally filtered to remove the solid part. To the filtrate compound of formula (4) is added. The mixture is stirred, preferably under an inert atmosphere like nitrogen or argon, at a temperature between 100° C. and 120° C. for between 1.5 and 5 hours. The reaction progress can be monitored by any suitable analytical method for example by HPLC or GC. When the reaction is completed, to the mixture a base was added. The base can be for example an hydroxide, such as NaOH or KOH or LiOH or an alkoxide such as a methoxide or an ethoxide or a butoxide. The molar ratio between the base and the compound of formula (4) can be between 0.15:1 and 0.3:1. The mixture is then stirred for between 1.5 and 5 hours. The mixture was cooled to a temperature between 30° C. and 50° C., preferably to a temperature between 38° C. and 42° C. The reaction progress can be monitored by any suitable analytical method for example by HPLC or GC. When the reaction is completed, to the mixture a mixture of the base and water and an alcohol such as methanol or ethanol or propanol is added. The concentration of the base in the mixture of water and the alcohol can be between 0.01 g/g and 0.03 g/g. The weight ratio between water and the alcohol can be between 2.8:1 and 3.5:1. The base solution in the mixture water and the alcohol can be added in the course of between 5 and 30 minutes. The mixture is then stirred for between 5 and 30 minutes at a temperature between 35° C. and 45° C. The mixture is then cooled to a temperature between 15;C and 25° C. and stirred at this temperature for between 45 and 120 minutes to obtain a suspension. Obtained solid was filtered off to obtain solid form of compound of formula (12),

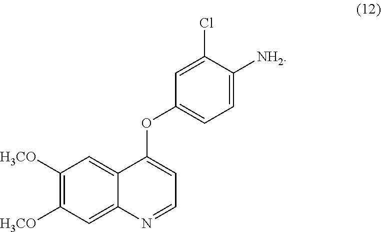 [SMILES: COc1cc2nccc(Oc3ccc(N)c(Cl)c3)c2cc1OC]

Solid form of compound of formula (12) can be recrystallized by a process comprising:
1. Dissolving solid form of compounf of formula (12) in a in a mixture of a base, water, N,N-dimethylacetamide and methanol;
2. Isolating of the solid form

The concentration of compound of formula (12) in the mixture water, N,N-dimethyl-acetamide and methanol can be between 0.04 g/g and 0.08 g/g. The volume ratio between N,N-dimethylacetamide and methanol can be between 4:1 and 5:1. The volume ratio between water and methanol can be between 2:1 and 3:1. The base can be selected for example from a hydroxide such and NaOH or KOH or LiOH. The concentration of the base in the solvent mixture water, N,N-dimethylacetamide and methanol can be between 0.007 g/g and 0.02 g/g. Compound of formula (12) is dissolved in the mixture of water, N,N-dimethylacetamide and methanol at a temperature between 70° C. and 90° C. The solution is cooled to a temperature between 35° C. and 45° C. and stirred at this temperature for between 5 and 30 minutes. The mixture is cooled to a temperature between 15° C. and 25° C. and stirred at this temperature for between 50 and 120 minutes. Obained suspension was filtered to provide a solid form of compound of formula (12).

Tivozanib, compound of formula (10), a salt or a solvate thereof, can be obtained from compound of formula (12) by reacting compound of formula (12) with compound of formula (13). The reaction can be performed in a solvent selected for example from N,N-Dimethylformamide or Dimethylacetamide or N-Methyl pyrrolidone. The concentration of compound of formula (12) in the solvent can be between 0.1 and 0.3 g/ml. The concentration of compound of formula (3) in the solvent can be between 0.07 and 0.2 g/ml. The molar ration between compound of formula (12) and compound of formula (3) can be between 0.7:1 and 1:1. The reaction is performed in the presence of pyridine or a salt thereof, preferably pyridine HCl. The molar ratio between compound of formula (12) and pyridine or a salt thereof can be between 6:1 and 7:1. Compound of formula (12) is mixed with compound of formula (3), pyridine or a salt thereof and the solvent. The mixture is heated to a temperature between 90° C. and 120° C. and stirred at this temperature for between 20 and 60 minutes. The reaction progress can be monitored by any suitable analytical method for example by HPLC or GC. After the reaction is completed, the mixture is cooled to a temperature between 55° C. and 65° C. and can be optionally filtered off. The temperature of the filtrate was set to a temperature between 40° C. and 50° C. To the mixture ethanol, water and hydrochloric acid was added. Hydrochloric acid is preferably 36% solution in water. Volume ratio between water and ethanol can be between 0.2:1 and 0.3:1. Molar ratio between hydrochloric acid and compound of formula (12) can be between 1:1.1 and 1:1.7, preferably it is between 1:1.3 and 1:1.4. The mixture can be optionally seeded by a solid form of compound of formula (10). The mixture is then stirred at a temperature between 40° C. and 50° C. for between 1.5 and 5 hours. The mixture is cooled to a temperature between 10° C. and 20° C. and stirred at this temperature for between 2.5 and 5 hours to provide solid form of Tivozanib, compound of formula (10) a salt or a solvate thereof. Tivozanib, compound of formula (10) is preferably prepared in form of monohydrate solvate of HCl salt.

Tivozanib, compound of formula (10), a salt or a hydrate thereof, preferably monohydrate solvate of HCl salt, can be crystallized by a process comprising:
1. Dissolving compound of formula (10), a salt or a solvate thereof, preferably monohydrate solvate of HCl salt in N,N-Dimethylformamide;
2. Cooling the mixture;
3. Adding a mixture of ethanol and water;
4. Isolating the solid form.

Compound of formula (10), a salt or a solvate thereof, preferably monohydrate solvate of HCl salt is dissolved in N,N-Dimethylformamide at a temperature between 70° C. and 90° C. and was stirred at this temperature for between 5 and 40 minutes. The concentration of compound of formula (10) can be between 0.2 g/ml and 0.4 g/ml. Then the mixture was cooled to a temperature between 15° C. and 30° C. To the mixture a mixture of ethanol and water was added. The volume ratio between added water/ethanol mixture and N,N-Dimethyl-formamide can be between 3:1 and 4:1. Obtained mixture can be optionally seeded with solid form of compound of formula (10), a salt or solvate thereof, and stirred for between 1 nad 10 hours to obtain a suspension. Obtained solid form of compound of formula (10), a salt or solvate thereof is filtered off Obtained solid form of compound of formula (10), a salt or solvate thereof can be optionally dried.

Compound of formula (13) can be prepared by a process comprising reacting compound of formula (14) with compound of formula (15) in a presence of pyridine or a salt thereof,

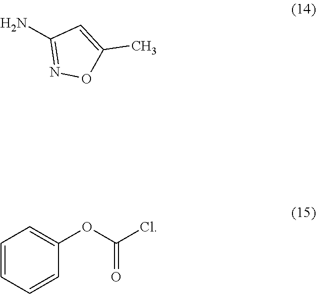

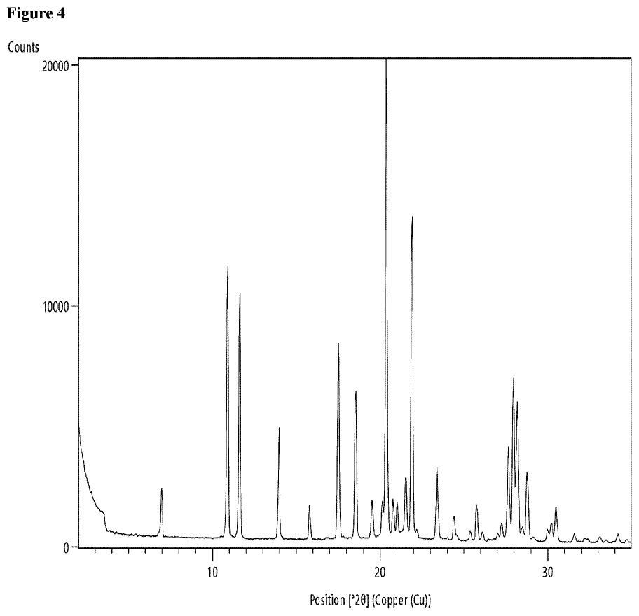

The reaction is performed in a solvent for example selected from N,N-Dimethyl-formamide or Dimethylacetamide or N-Methyl pyrrolidone or tetrahydrofurane. The concentration of compound of formula (14) in the solvent can be between 0.07 g/ml and 0.15 g/ml. The concentration of compound of formula (15) in the solvent can be between 0.10 g/ml and 0.25 g/ml. Molar ratio between compound (14) and compound (15) can be between 1:1 and 1:1.2. Compound of formula (14) is mixed with the solvent, the mixture was cooled to a temperature between −5° C. and 5° C. To the mixture pyridine or a salt thereof was added. The molar ratio between pyridine and compound of formula (14) can be between 0.5:1 and 0.8:1. To the mixture compound of formula (15) was added in the course of between 20 and 45 minutes at a temperature between −5° C. and 5° C. Mixture was stirred at a temperature between −5° C. and 5° C. for between 15 and 45 minutes. The reaction progress can be monitored by any suitable analytical method for example by HPLC or GC. After the reaction is finished water is added in the course of between 20 and 60 minutes. The volume ratio between water and the solvent used for reaction between compounds (14) and (15) can be between 1:1 and 1:1.2. Obtained suspension was cooled to a temperature between −5° C. and 5° C. and stirred at this temperature for between 20 and 50 minutes. Obtained suspension was filtered off to provide solid form of compound of formula (13). Obtained solid form of compound of formula (13) can be washed with a mixture of n-heptane and isopropyl alcohol. The volume ratio between n-heptane and isopropyl alcohol can be between 1:1 and 1.3:1. XRPD pattern of prepared solid corresponds to XRPD pattern depicted in FIG. 4. By using the process described above, obtained solid form of Compound of formula (3) contains less than 1% (wt/wt) of solvents. Using of the solid form of compound (3) comprising less than 1% (wt/wt) of solvents for preparation of Tivozanib, a last or a solvate thereof, improves the yield and purity of Tivozanib.

Solid form of compound of formula (13) can be crystallized by a process comprising:
1. Dissolving solid form of compound of formula (13) in N,N-Dimethylformamide;
2. Adding of water;
3. Isolating the solid form.

Concentration of compound of formula (13) in N,N-Dimethylformamide can be between 0.2 and 0.5 g/ml. Compound of formula (13) is dissolved in N,N-Dimethyl-formamide at atemperature between 30° C. and 50° C. To the solution water was added in the course of between 5 and 30 minutes. The volume ratio between water and N,N-Dimethyl-formamide can be between 2.5:1 and 3:1. Mixture was cooled to a temperature between −5° C. and 5° C. and stirred at this temperature for between 15 and 45 minutes. Obtained suspension was filtered off to provide solid form of compound of formula (13). Obtained solid form of compound of formula (13) can be washed with a mixture of n-heptane and isopropyl alcohol. The volume ratio between n-heptane and isopropyl alcohol can be between 1:1 and 1.3:1. XRPD pattern of prepared solid corresponds to XRPD pattern depicted in FIG. 4.

By using the process described above, obtained solid form of Compound of formula (3) contains less than 1% (wt/wt) of solvents. Using of the solid form of compound (3) comprising less than 1% (wt/wt) of solvents for preparation of Tivozanib, a last or a solvate thereof, improves the yield and purity of Tivozanib.

Cabozantinib, compound of formula (1), or Tivozanib, compound of formula (10), or salt or a solvate thereof, prepared according to presented invention, can be also processed into a suitable pharmaceutical formulation. In the pharmaceutical formulation it can be mixed with pharmaceutically acceptable adjuvants, diluents or carriers. The amount of Cabozantinib or Tivozanib or a salt or a solvate thereof in the formulation depends on the condition and a patient to be treated. The pharmaceutical formulation can be if form of a solid oral formulation, for example a capsule, a pill, a powder or a granule. In the formulation Canbozantinib or Tivozanib or a salt or a solvate thereof can be mixed with one or more additives such as fillers or extenders or binders or wetting agents or disintegrating agents or absorbents or lubricants or buffering agents. The formulation in a form of a tablet or a dragee or a capsule or a pill or a granule can be coated with a coating or shell such as enteric or other coating. The oral formulation can be in a form of an oral emulsion or a solution or a suspension or a syrup. The formulation can contain suitable additives such as diluent(s) or wetting agent(s) or emulsifying agent(s) or suspending agent(s) or sweetening agent(s) or flavouring agent(s). The examples of suitable additive(s) are known to those skilled in the art.

The suitable pharmaceutical formulation can be in a parenteral form such as an injection or an infusion or an injectable depot or in a liposomal form comprising pharma-ceutically acceptable aqueous or non-aqueous solution(s) or dispersion(s) or emulsions. The pharmaceutical formulation can be also in a form of a powder for reconstitution into an injection or infusion. The formulation can further comprise additives such as preservative(s) or wetting agent(s) or emulsifying agent(s) or dispersing agent(s) or antibacterial or anti-fungal agents. The examples of suitable additive(s) are known to those skilled in the art.

The suitable pharmaceutical formulation can be in a form suitable for rectal or vaginal administration further comprising suitable additive(s). The examples of suitable additive(s) are known to those skilled in the art.

Cabozantinib or Tivozanib or a salt or a solvate thereof can be used for the treatment of conditions treatable with Cabozantinib or Tivozanib or a salt thereof.

The invention will be further described with reference to the following examples.

### EXAMPLES

XRPD spectrum was obtained using the following measurement conditions:
Panalytical Empyrean diffractometer with Θ/2Θ geometry (transmition mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 | Å (Kα1) |
| Divergence slit: | ½° | |
| Antiscatter slit: | ½° | |
| Soller slit: | 0.02 | rad |
| Detector slit: | 7.5 | mm |
| Rotation speed: | 30 | rpm |

### Example 1 Preparation of 4-chloro-6,7-dimethoxyquinoline (Compound of formula 4)

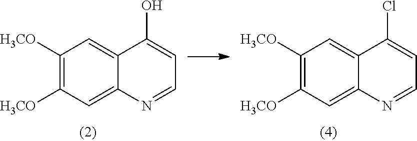 [SMILES: COc1cc2nccc(O)c2cc1OC ; COc1cc2nccc(Cl)c2cc1OC]

200 g of 6,7-dimethoxyquinolin-4-ol (Compound of formula (2)) suspended in 1059 ml of N,N-dimethylformamide (DMF). The mixture was heated to 80° C. and distilled with vacuum. 300 ml of solvent was distilled off After distillation, temperature was cooled to 70° C. To the mixture 320 ml of N,N-dimethylformamide and 114.8 g of triethylamine were added. Then 207 g of benzenesulfonyl chloride (10 min) was added to the mixture during 10 minutes.

Reaction mixture was stirred at 70° C. for 2.5 hours. Then, reaction mixture was filtered through celite. The filtrate was cooled down to 20° C. and a solution of 15 g of NaOH in 1485 ml of water was added to reaction mixture. Mixture was stirred at 30° C. for 30 minutes and then was cooled down to 10° C. At this temperature, suspension was stirred for 1 hour. Obtained suspension was filtered off and obtained mass was washed with 500 ml of water and then by 200 ml of n-heptane and dried in vacuum dryer at 70° C. overnight to provide 193.64 g of compound of formula (4) in purity 99.7% (HPLC IN).

### Example 2 Preparation of 4-chloro-6,7-dimethoxyquinoline (Compound of formula 4)

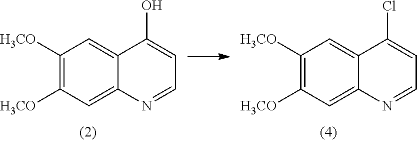 [SMILES: COc1cc2nccc(O)c2cc1OC ; COc1cc2nccc(Cl)c2cc1OC]

100 g of 6,7-dimethoxyquinolin-4-ol (Compound of formula (2)) suspended in 530 ml of N,N-dimethylformamide at a temperature between 20° C. and 25° C. The mixture was heated to 70° C. and distilled with vacuum. 250 ml of solvent was distilled off.

To the mixture 57.4 g of triethylamine followed by 117 g of 4-methylbenzenesulfonyl chloride were added with stirring. Reaction mixture was stirred at 70° C. for 2.5 hours. The mixture was cooled to 50° C. To the mixture 750 ml of water was added. To the mixture 4M water solution of NaOH was added (solution=19.94 g NaOH+101 g water). Resulting suspension was stirred at (50 to 52) ° C. for 30 minutes. Suspension was cooled down to a temperature between 20° C. and 25° C. during 1 hour. Suspension was filtered off and obtained solid was washed with 500 ml of water. Solid Compound of formula (4) was dried in vacuum dryer at 70° C. to provide 91.92 g of Compound of formula (4), purity 99.7% (HPLC IN).

### Example 3 Preparation of 4-((6,7-dimethoxyquinolin-4-yl)oxy)aniline (Compound of formula 6)

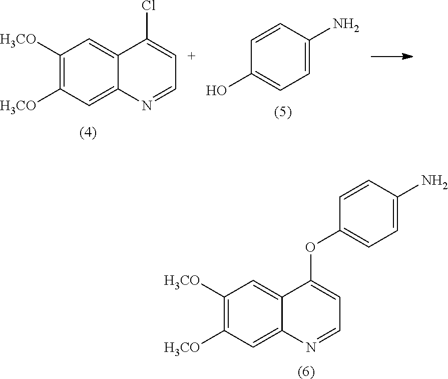

5 g of compound of formula (4) was mixed with 270 ml of N,N-dimethylacetamide, 1.925 g of potassium hydroxide and 3.66 g of 4-aminophenol (Compound of formula (5)). (3.66 g, 33.5 mmol) and stirred for 15 minutes. Suspension was stirred for 15 minutes and inertized by nitrogen or argon (3 L/hr). Temperature of the mixture was set to 110° C. and stirred at this temperature for 2.5 hours. The mixture was cooled down to 40° C. To the mixture 200 ml of methanol was added. To the mixture a solution of KOH and water (0.47 g of KOH+49.53 g of Water) was added over 5 minutes.

The mixture was stirred at 40° C. for 10 minutes. Then, the mixture was cooled to 20° C. and stirred at this temperature for 1 hour. Obtained suspension was filtered off, obtained solid was collected by filtration, washed successively with mixture of 4 ml of N,N-dimethyl-acetamide and 2 ml of water, then with 3 ml of water and then with 5 ml of tetrahydrofurane, dried on filter to provide 4.45 g of Compound of formula (6), purity 99.7% (HPLC IN).

### Example 4 Preparation of 1-((4-fluorophenyl)carbamoyl)cyclopropane-1-carboxylic acid (Compound of formula (7))

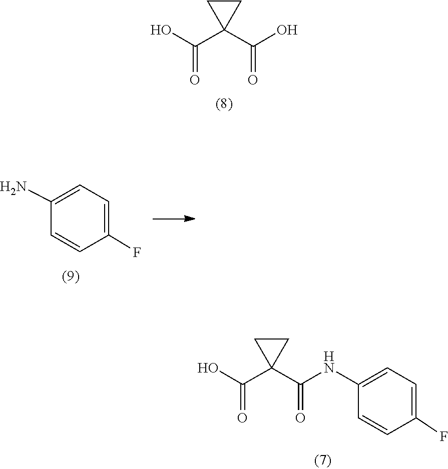 [SMILES: O=C(O)C1(C(=O)O)CC1 ; Nc1ccc(F)cc1 ; O=C(O)C1(C(=O)Nc2ccc(F)cc2)CC1]

To a pre-cooled solution (0° C.) of 10 g of cyclopropane-1,1-dicarboxylic acid (Compound of formula (8)) in 40 ml of 2-methyl tetrahydrofurane (MeTHF) was added dropwise 6.13 ml of thionyl chloride at 0° C. Resulting mixture was stirred for 2 hours at 0° C.

To obtained mixture, a mixture prepared from 6.92 ml of 4-Fluroaniline in 25 ml of 2-methyl tetrahydrofurane was added dropwise (temperature kept below 5° C.). Resulting mixture was stirred for additional 40 min at 0° C.

Then, a mixture of 9.49 g KOH in 60 ml of water was added slowly (in the course of 30 minutes) (temperature kept below 10° C.), and then the mixture was heated to 23° C.

The phases were separated. Organic phase was washed with 50 ml of 10% aqueous solution of NaCl. Phases were separated, organic phase was diluted with 50 ml of toluene. Volatile fraction was removed by evaporation in vacuo at 50° C., 50 ml of the solvent was distilled off (precipitation of material was observed). Formed suspension was cooled down to 0° C. and then was stirred for 2 hours at this temperature. The solid mass was collected by filtration, washed with 10 ml of toluene and suction dried on air for 2 hrs to give 9.01 g of Compound of formula (7) in purity 89.6% (HPLC IN).

This material (9.01 g) was dissolved in the mixture of 140 ml of isopropyl acetate (i-PrOAc) and 60 ml of water. The mixture was stirred for 5 minutes at 23° C. The phases were separated, organic phase was dried over MgSO₄. The solvent was removed in vacuo at 45° C. until precipitation of material was observed (100 ml of solvent was distilled off). Formed suspension was cooled down to 23° C. and stirred for 4 hours at 23° C. The solid was collected by filtration, washed with 10 ml of i-PrOAc and suction dried on air for 2 hrs to give 8.31 g of Compound of formula (7) in 100% purity (HPLC IN).

### Example 5 Preparation of 1-((4-fluorophenyl)carbamoyl)cyclopropane-1-carboxylic acid (Compound of formula (7))

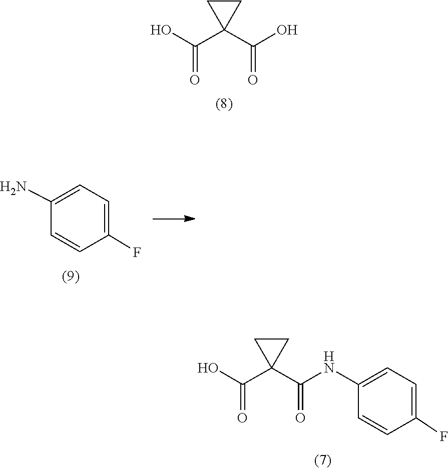 [SMILES: O=C(O)C1(C(=O)O)CC1 ; Nc1ccc(F)cc1 ; O=C(O)C1(C(=O)Nc2ccc(F)cc2)CC1]

To a pre-cooled solution (0° C.) of 30 g of cyclopropane-1,1-dicarboxylic acid (Compound of formula (8)) in 120 ml of methyl tert-butyl ether (MTBE) 18.4 ml of thionyl chloride was added dropwise at 0° C. Resulting mixture was stirred for 2 hours at this temperature.

To obtained resulting mixture, a mixture prepared from 65.5 ml of 4-fluroaniline (Compound of formula (9)) in 120 ml of MTBE was added dropwise (temperature kept below 25° C.). The mixture was stirred for additional 40 minutes at 0° C. To the mixture pre-cooled (5° C.) 160 ml solution of 5M aqueous NaOH was added slowly (in the course of 30 minutes, temperature kept below 25° C.) and then, the mixture was stirred for 5 min at 25° C.

The phases were separated, aqueous phase was discarded and organic phase was washed with 1 M NaOH (200 mL). The phases were separated, organic phase was discarded and aqueous phase was acidified with 35% aqueous HCl (23 mL) to pH=1.8 (precipitation of material was observed). The formed suspension was stirred 10 min at 23° C. and then, the solid was collected by filtration, washed with 50 ml of 0.01 M HCl and suction dried on air for 30 min, then dried in vacuum oven (70° C., 100 mbar, N2 strip) to give 35 g of compound of formula (7) (68% yield, 99.82% purity, 95.6% assay, HPLC IN).

### Example 6 Preparation of 1-((4-fluorophenyl)carbamoyl)cyclopropane-1-carboxylic acid (Compound of formula (7))

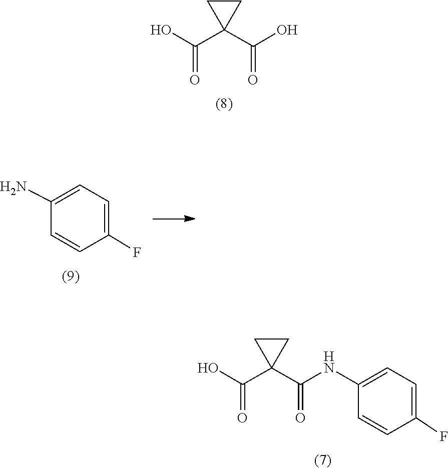 [SMILES: O=C(O)C1(C(=O)O)CC1 ; Nc1ccc(F)cc1 ; O=C(O)C1(C(=O)Nc2ccc(F)cc2)CC1]

To the mixture of 35 g SOCl₂ in 50 g of methyl tert-butyl ether (MTBE) 30 g of compound of formula (8) was added at a temperature between 20° C. and 25° C. The mixture was stirred at this temperature for 1 hour. Obtained mixture was drop-wise added to pre-cooled (0-5° C.) two-phase mixture of 30.8 g of compound of formula (9) in 50 g of MTBE and 18.6 g of NaOH in 100 g of water (temperature of the mixture was maintained under 40° C.). The mixture was stirred at 35° C. for 1 hour. pH of the mixture was set to 12 by 250 g of 5N solution of NaOH. Phases were separated. To water phase 100 g of concentrated HCl was added to set pH of the mixture to 1 (temperature of the mixture was maintained under 45° C.). The mixture was then cooled to 0° C. and obtained suspension was filtered. Obtained solid was dried under vacuum at 70° C. to provide 43 g of Compound of formula (7), purity 99.6% (HPLC IN).

### Example 7 Preparation of 1-((4-fluorophenyl)carbamoyl)cyclopropane-1-carboxylic acid (Compound of formula (7))

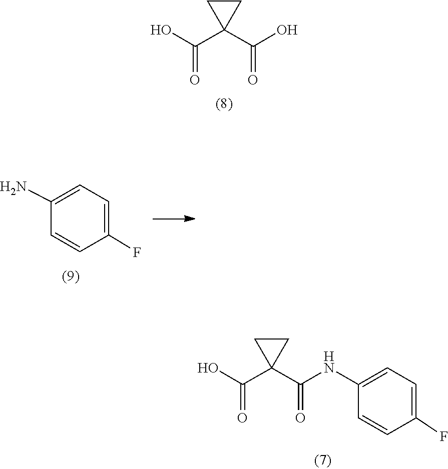 [SMILES: O=C(O)C1(C(=O)O)CC1 ; Nc1ccc(F)cc1 ; O=C(O)C1(C(=O)Nc2ccc(F)cc2)CC1]

To the mixture of 35 g SOCl₂ in 50 g of methyl tert-butyl ether (MTBE) 30 g of compound of formula (8) was added at a temperature between 35° C. and 40° C. The mixture 25 was stirred at this temperature for 1 hour. Obtained mixture was drop-wise added to pre-cooled (0-5° C.) two-phase mixture of 30.8 g of compound of formula (9) in 50 g of MTBE and 18.6 g of NaOH in 100 g of water (temperature of the mixture was maintained under 40° C.). The mixture was stirred at 35° C. for 1 hour. pH of the mixture was set to 12 by 250 g of 5N solution of NaOH. Phases were separated. To water phase approx. 100 g of concentrated HCl was added to set pH of the mixture to 1 (temperature of the mixture was maintained under 45° C.). The mixture was then cooled to 0° C. and obtained suspension was filtered. Obtained solid was dried under vacuum at 70° C. to provide 43 g of Compound of formula (7), purity 99.6% (HPLC IN).

### Example 8 Preparation of N-(4-((6,7-dimethoxyquinolin-4-yl)oxy)phenyl)-N′-(4-fluorophenyl)cyclopropane-1,1-dicarboxamide (compound of formula (1))

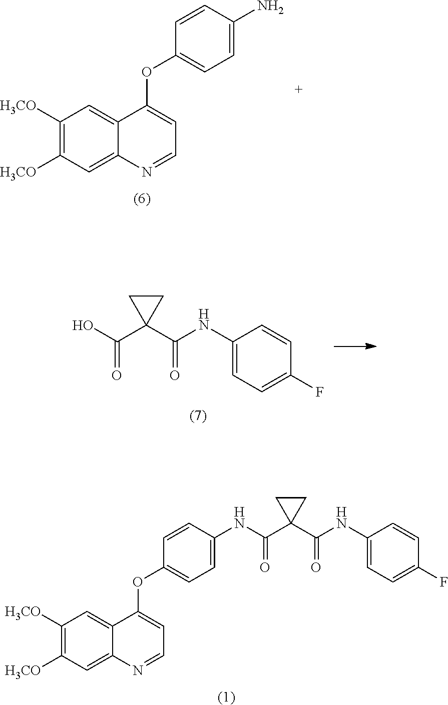 [SMILES: COc1cc2nccc(Oc3ccc(N)cc3)c2cc1OC ; O=C(O)C1(C(=O)Nc2ccc(F)cc2)CC1 ; COc1cc2nccc(Oc3ccc(NC(=O)C4(C(=O)Nc5ccc(F)cc5)CC4)cc3)c2cc1OC ; "(1)"]

The reaction was perfomed under Argon atmosphere.

5 g of Compound of formula (6) was mixed with 4.14 g of Compound of formula (7), 3.26 g of 2-chloro-4,6-dimethoxy-1,3,5-triazine (CDMT) and 90 ml of tetrahydrofurane at 20-25° C. with stirring. To the mixture 2.041 ml of 4-methylmorpholine was added at once at 20-25° C. with stirring. The resulting suspension was stirred at 20-25° C. for 4 hours. The mixture was washed with 60 ml of 15% aqueous NaCl at 60° C. (stirring for 15 min). The phases were separated. Organic phase was washed twice with 60 ml of saturated aqueous NaHCO₃ at 60° C., stirring for 15 minutes, phases were separated. Organic phase was cooled to between 25 and 30° C. To the organic phase 500 mg of active carbon was added and the mixture was stirred at 20-25° C. over weekend. The mixture was filtered, dried over MgSO₄ and concentrated (60° C., 200-->40 mbar) to dryness. 7.46 g of Compound of formula (1) 7.46 g, 96.69% purity (HPLC in) was obtained.

### Example 9 Preparation of N-(4-((6,7-dimethoxyquinolin-4-yl)oxy)phenyl)-N′-(4-fluorophenyl)cyclopropane-1,1-dicarboxamide (compound of formula (1))

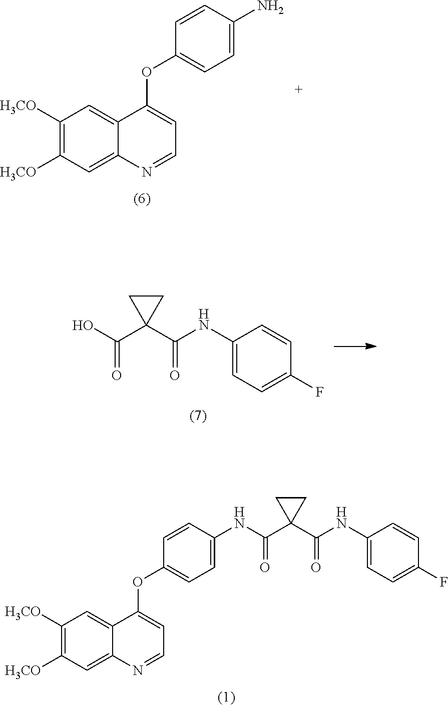 [SMILES: COc1cc2nccc(Oc3ccc(N)cc3)c2cc1OC ; O=C(O)C1(C(=O)Nc2ccc(F)cc2)CC1 ; COc1cc2nccc(Oc3ccc(NC(=O)C4(C(=O)Nc5ccc(F)cc5)CC4)cc3)c2cc1OC ; "(1)"]

4.9 g of compound of formula (7) were added to a solution of 3.6 g of thionyl chloride in 56.2 ml of tetrahydrofurane (THF). The mixture was stirred at 62° C. for 2 hours to obtain a mixture. The mixture was dropwise added during 10 minutes to a mixture of compound of formula (6) with 56.2 ml of tetrahydrofurane, 2.4 g of NaOH and 50 ml of water. To obtain mixture 5 g of 5N NaOH aqueous solution was added. The phases were separated and the organic mixture was washed twice with 50 ml of saturated solution of NaCl. Organic phase was dried over MgSO₄ and concentrated to dryness to obtain compound of formula (1).

### Example 10: Preparation of 2-chloro-4-((6,7-dimethoxyquinolin-4-yl)oxy)aniline

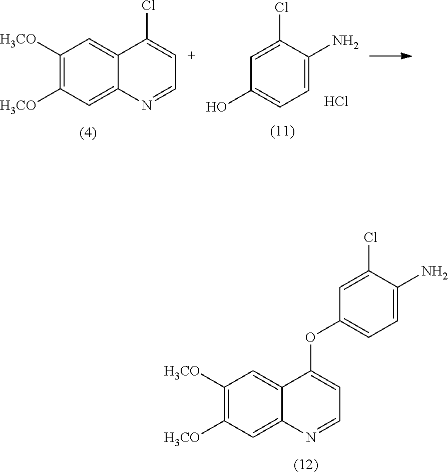 [SMILES: COc1cc2c(cc1OC)C(Cl)=[C+]C=N2 ; Nc1ccc(O)cc1Cl ; Cl ; COc1cc2nccc(Oc3ccc(N)c(Cl)c3)c2cc1OC]

The reaction was performed under nitrogen. 137 g of HCl salt of compound (11) was dissolved in 982 ml N,N-Dimethylacetamide at 20-25° C. The solution was cooled down to 0° C. 77 g of triethylamine was added to mixture by dropping funnel over 10 minutes. The mixture was cooled down to 0-5° C. and stirred for 20 minutes. The solid material was removed by filtration through kieselguhr and washed by 213 ml of N,N-Dimethylacetamide. To the filtrate 100 g of compound (4) was added at 20° C. The mixture was heated to 110° C. To the solution 41.8 g of KOH was added. The mixture was stirred for 2 hours at 110° C. Then, 5.56 g of KOH was added and the reaction mixture and stirred another 2 hours.

The mixture was cooled down to 40° C. A mixture of 12 g KOH, 588 g of H₂O and 200 g of MeOH was added in the course of 10 minutes. Suspension was stirred 10 minutes at 40° C., cooled down to 20° C. and stirred at this temperature for 1 hour. Obtained solid for filtered off, washed by 3×400 ml of water and cooled (10° C.) mixture of H₂O+MeOH (300 ml H₂O+252 ml MeOH). Product was dried on filter, overnight to provide 129 g (87% of theoretical yield) of compound of formula (12) in >99% purity.

Solid form of compound of formula (12) can be crystallized by following process:

129 g of compound of formula (12) was dissolved in 1523 ml of N,N-Dimethylacetamide at 80° C. Solution was cooled down to 40° C. A mixture of KOH+H₂O +MeOH (31.15 g KOH+763.28 g H₂O+259.6 g MeOH) was added over 12 minutes. Obtained suspension was stirred 10 minutes at 40° C. The mixture was cooled down to 20° C. and stirred at this temperature for 1 hour. Obtained suspension was filtered off and obtained solid was washed by 3×400 ml Water and a cooled mixture (10° C.) of H₂O+MeOH (300 ml H₂O+252 ml MeOH). Obtained solid was dried in vacuum dryer at 40° C., 3 hours to provide solid form of compound of formula (12) in 95% yield and >99% purity.

### Example 11: Preparation of Tivozanib HCl Monohydrate

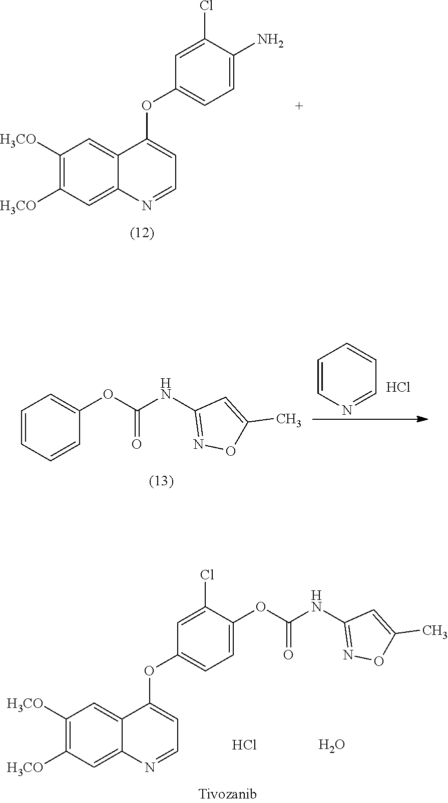

100 g of compound (12) was mixed with 882 ml of N,N-Dimethylformamide. 5.69 g of Pyridine hydrochloride and 79 g of compound (13) were added. Reaction mixture was heated up to 110° C. and stirred 30 minutes at this temperature. The mixture was cooled down to 60° C. and filtered. Filtrate was heated up to 45° C. To the mixture 983 ml of Ethanol, 35 ml of 36% Hydrochloric acid and 212 ml of Water were added.

18 ml of the mixture was stirred in a vial for 30 minutes at 20-25° C. to provide seeds that can be optinally used in next steps.

The mixture can be optionally seeded. Mixture was stirred 2 hours at 45° C. Then, mixture was cooled down to 15° C. and stirred 3 hours at this temperature. Solid Tivozanib HCl monohydrate was collected by filtration and washed by 748 ml of cooled (10° C.) Ethanol. Product was dried on filter 4 hours to provide 124 g (81% of theoretical yield) of product in >99% of purity.

Solid form of Tivozanib HCl monohydrate can be crystallized by following process.

124 g of Tivozanib HCl monohydrate was dissolved in 525 ml of N,N-Dimethyl-formamide at 85° C. and stirred at this temperature for 10 minutes. The mixture was cooled down to 20° C. and 1572 ml of Ethanol and 248 ml of Water (248 ml) were added. Prepared mixture was stirred for 30 minutes at 20° C. The mixture can be optionally seeded. The 20 mixture was stirred 4 hours at 20° C. Obtained suspension was filtered off, obtained solid was washed by 748 ml of cooled (10° C.) Ethanol.

Solid Tivozanib HCl monohydrate was dried on filter 2 hours, then in vacuum dryer at 45° C. for 3 hours to provide 94 g (76% of theoretical yield) in >99% of purity.

### Example 12: Preparation of phenyl (5-methylisoxazol-3-yl)carbamate

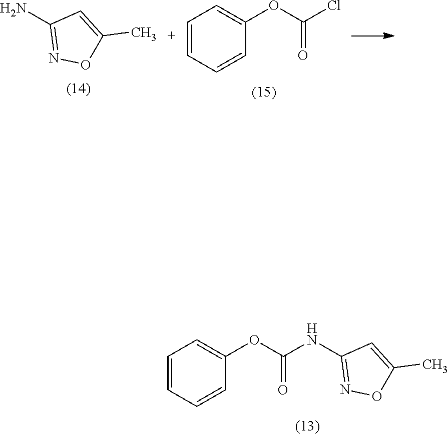

100 g of compound of formula (14) was dissolved in 1059 ml of N,N-Dimethyl-formamide at 20° C. Solution was cooled down to 0° C. and then 123 ml of Pyridine was added at once. When the mixture was re-cooled down to 0° C. 128 ml of compound of formula (15) was added at 0° C. by dropping funnel over 35 minutes Reaction mixture was stirred at 0° C. for 30 minutes. To the mixture 1100 ml of water was added slowly by dropping funnel over 30 minutes. Suspension was cooled down to 0° C. and stirred at this temperature for 30 minutes. Obtained suspension was filtered off. Obtained solid was washed by 2×500 ml of water and cooled (10° C.) mixture of DMF+water (228 ml DMF+215 ml water) and then cooled (10° C.) mixture of n-heptane+isopropyl alcohol (1125 ml n-heptane +980 ml isopropyl alcohol). The solid was dried on filter for 30 minutes, then dried in vacuum dryer at 45° C. for 8 hours. 209 g (94% of theoretical yield) of solid compound (13) was obtained in >99% yield. XRPD pattern of prepared solid corresponds to XRPD pattern depicted in FIG. 4.

Solid compound (13) can be crystallized by following process.

209 g of Solid compound (13) was dissolved in 885 ml of N,N-Dimethylformamide at 40° C. To the mixture 2299 ml of Water in the course of 10 minutes were added. Suspension was cooled down to 0° C. and stirred at this temperature for 30 minutes. Obtained suspension was filtered off and solid form of compound (13) was washed by 2×500 ml of Water and cooled (10° C.) mixture of DMF+H₂O (228 ml DMF+215 ml H₂O) and then cooled (10° C.) mixture of n-heptane+isopropyl alcohol (1125 ml n-heptane+980 ml isopropyl alcohol). The solid was dried on filter for 30 minutes, then was dried in vacuum dryer at 45° C. for 8 hours to provide 194 g (93% of theoretical yield) of solid compound (13) in >99% purity. XRPD pattern of prepared solid corresponds to XRPD pattern depicted in FIG. 4.

1. A process for the preparation of a compound of formula (1) (“Cabozantinib”); or a compound of formula (10) (“Tivozanib”) or a salt or a solvate thereof: 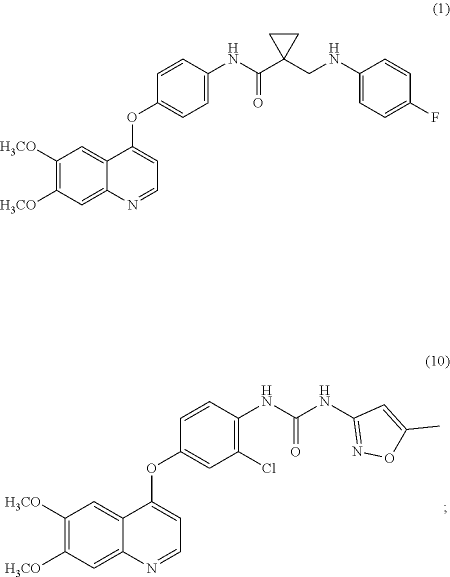 [SMILES: COc1cc2nccc(Oc3ccc(NC(=O)C4(CNc5ccc(F)cc5)CC4)cc3)c2cc1OC ; COc1cc2nccc(Oc3ccc(NC(=O)Nc4cc(C)on4)c(Cl)c3)c2cc1OC]
the process comprising:
a) reacting a compound of formula (2) with a compound of formula (3) in a solvent selected from N,N-dimethylformamide or N,N-dimethylacetamide or N-methyl pyrrolidone to obtain a compound of formula (4): 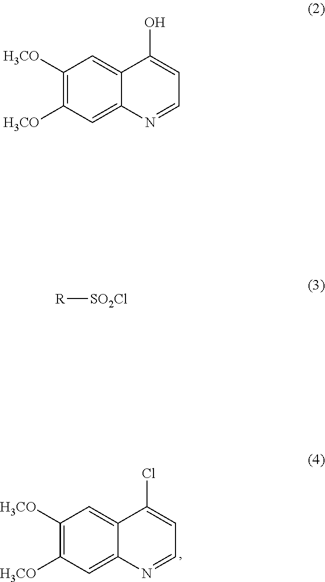
R is selected from R is selected from C₁ to C₇ linear or branched optionally subsituted alkyl or aryl;
b) transforming the compound of formula (4) into Cabozantinib or Tivozanib or a salt or a solvate thereof.

2. The process according to claim 1 wherein the solvent is N,N-dimethylformamide.

3. The process according to claim 1 wherein the reacting in step a) is perfomed at a temperature between 70° C. and 90° C.

4. The process according to claim 1 wherein the compound of formula (3) is selected from benzenesulfonyl chloride or 4-methylbenzene sulfonyl chloride or methanesulfonyl chloride or ethanesulfonyl chloride.

5. The process according to claim 4 wherein the compound of formula (3) is selected from benzenesulfonyl chloride or 4-methylbenzene sulfonyl chloride.

6. The process according to claim 1 wherein the step a) further comprises addition of a water solution of a base to precipitate a solid form of the compound of formula (4).

7. The process according to claim 4 wherein the base is selected from a hydroxide or a carbonate.

8. The process according to claim 1 wherein the step b) comprises:
a) reacting the compound of formula (4) with a compound of formula (5) to obtain a compound of formula (6): 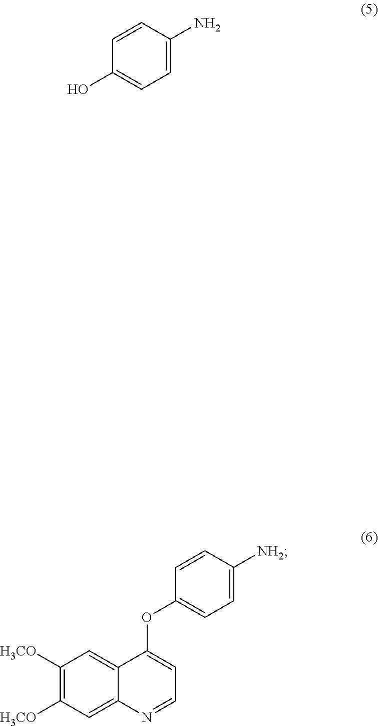 [SMILES: Nc1ccc(O)cc1 ; COc1cc2nccc(Oc3ccc(N)cc3)c2cc1OC]
b) reacting the compound of formula (6) with a compound of formula (7) to obtain the compound of formula (1):

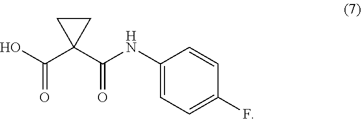 [SMILES: O=C(O)C1(C(=O)Nc2ccc(F)cc2)CC1]

9. The process of claim 8 wherein the compound of formula (7) is prepared by reacting a compound of formula (8) and a compound of formula (9):

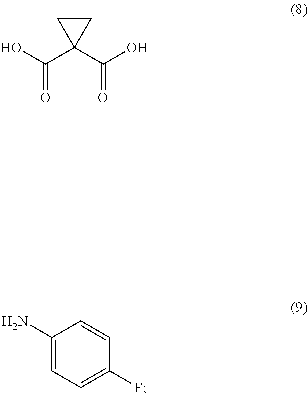 [SMILES: O=C(O)C1(C(=O)O)CC1 ; Nc1ccc(F)cc1]

10. A process for preparation of a compound of formula (7): 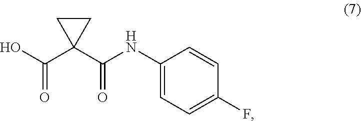 [SMILES: O=C(O)C1(C(=O)Nc2ccc(F)cc2)CC1]
the process comprising:
a) mixing a compound of formula (8) with an acyl chloride forming agent in a solvent to obtain a mixture: 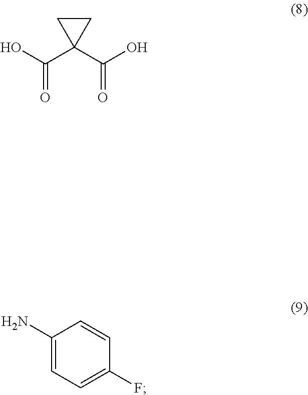 [SMILES: O=C(O)C1(C(=O)O)CC1 ; Nc1ccc(F)cc1]
b) adding the mixture into a two-phases system of a water immiscible solvent, a water solution of a base and compound of formula (9);
c) setting pH of a mixture obtained in step b) between 11 and 13;
d) separating the phases;
e) setting pH of water phase obtained in step d) between 1 and 2 to provide compound of formula (7).

11. The process according to claim 10 wherein the acyl chloride forming agent is selected from thionyl chloride or oxalyl dichloride.

12. The process according to claim 10 wherein the solvent in step a) is selected from tetrahydrofurane or 2-methyl tetrahydrofurane.

13. The process according to claim 10 wherein the water immiscible solvent is an ether.

14. The process according to claim 13 wherein the ether is methyl tert-butyl ether or tetrahydrofurane.

15. The process according to claim 10 wherein the base in step b) is selected from a hydroxide or a carbonate.

16. The process according to claim 1 further comprising transforming the compound of formula (1) into a salt or a solvate of the compound of formula (1).
